SEQUENCE LISTING (5 sequences)

SEQ ID NO: 1 - protein, 13 aa, Unknown
  mzip peptide
SIYRRGARRWRKL

SEQ ID NO: 2 - protein, 13 aa, Artificial Sequence
  synthetic peptide
RLYRKRIWRSAGR

SEQ ID NO: 3 - DNA, 2058 bp, homo sapiens
gggcccggacctctgtactcctccgtccctacactccccgccccctgtcctgtcggccggaaggcaatttatagacgaggagcgcgagctcggagggacggataacagccccggcctcgcttcggctgcgtcgtagtcgagcagttgcccttccttctacggagggacgtgcgggcggcgcgtgtctcgtatttcttagacgcgactcctccgtcctcttctttcggcttagatagatggcggcccctcggtcttctacctccttcgacatggcacggttgccggtggagaaggttcggttcgcgaaattgtcctctcgcatgacgccagtcacgtcgctctcctataccccggagcgctccgttccgatgtccacgtagttgacgtttgacgaccaggtattcgcgacggtgccggagcagggcgactggacgtccttcgtatacctaagacagtacggaagggttctcggaggtcatctgctgttcttgctcctgcggctggaaggaaggctcctctgtctaccttaacgaatgtaaaggagtagggccttcgtactgtcgtaatttctgctgagcctcctggaattcggtcaatagctaccctacctaccttagttttagagagtccccgaacccgacgtcctgaaactggattagtctcagtagcccgcgccctcgatgcggttccaagaggaccacgccaacttcttcttactggtttaaatgcggtactttcaccacttctttctcgaccacgtactactgctcctataactgacccatgtctgtctcttcgtgcacaaactcgtccgtaggtcgttggggaaggaccagcctaatgtgaggacgaaggtctgctgttcagccaacaaggaccagtaactcatgcagttgccgcccctggactacaaggtgtacgtctccgtctccttcgagggactcctcgtgcggtccaagatgcgccggctctagacgtagcgggagttgaaggacgtgctctccccctagtagatgtccctggacttcgacctgttgcaggaggacctacgcctgcccgtgtagttcgagtgtctgatgccgtacacgttccttccggacccgggaccactgtgttgctcgtgaaagacgccttggggcttaatgtagcgggggctttaggacgcccctctcctcatgcccaagtcgcacctgaccacccgcgaccctcaggagtacaaactctactaccggcccgcgaggggcaagctgtagtagtggctgttgggcctgtacttgtgtctcctgatggaaaaggttcactaggacctcttcgggtaggcctagggggccaaggacaggcagtttcggagggtacaaaattttcctaaaaatttattcctggggtttctctccgagccgacggccggtgtctgacctaaaagactgtagttcagggtgcgcaagaaggcgtcgtatctgaccctgaacgacctcttcttcgtccgcgagggaggtaaggtcggtgtctagtgtctgctgatgccagacctgttgaaactgtgtgtcaagtggtcgctcgggcacgtcgactggggtctgctactcctacggtatttctcctagctggtcagtctcaagcttccgaaactcatatagttgggtaataacgacaggtggctcctcagccacactccggcgcacgcagagacagcacctgtgcgcactaactgggaaattgacataggaattggtggcgtatacgtacggtccgacccgtgccgaggctcccgccggtccctgtctgcgaacgcggctctggcgtctcccttcgcagtcgcccgcgacgaccctcgtcttgtcagggagtgtggaccgggccgtccgtcgaagcacgacctccttgaacgacgacacggacgcagcgccgcctaggcgcccctgggacggctcccccgacagtacgccaaaggttccacgtgtaaaaggtgcctttgtcttgagctacgtgactggacgaggcggtcctttcactcgcacatcgcaggactccttattttacaaggctactttttttttt

SEQ ID NO: 4 - DNA, 2058 bp, homo sapiens
  CDS (444)..(1670)
  Unsure (522)..(522)
  Unsure (522)..(522): r at position 522 is g or a
cccgggcctggagacatgaggaggcagggatgtgaggggcgggggacaggacagccggccttccgttaaatatctgctcctcgcgctcgagcctccctgcctattgtcggggccggagcgaagccgacgcagcatcagctcgtcaacgggaaggaagatgcctccctgcacgcccgccgcgcacagagcataaagaatctgcgctgaggaggcaggagaagaaagccgaatctatctaccgccggggagccagaagatggaggaagctgtaccgtgccaacggccacctcttccaagccaagcgctttaacaggagagcgtactgcggtcagtgcagcgagaggatatggggcctcgcgaggcaaggctacaggtgcatcaactgcaaactgctggtccataagcgctgccacggcctcgtcccgctgacctgcaggaagcatatggattctgtcatgccttcccaagagcctccagtagacgacaagaacgaggacgccgaccttccttccgaggagacaratggaattgcttacatttcctcatcccggaagcatgacagcattaaagacgactcggaggaccttaagccagttatcgatgggatggatggaatcaaaatctctcaggggcttgggctgcaggactttgacctaatcagagtcatcgggcgcgggagctacgccaaggttctcctggtgcggttgaagaagaatgaccaaatttacgccatgaaagtggtgaagaaagagctggtgcatgatgacgaggatattgactgggtacagacagagaagcacgtgtttgagcaggcatccagcaaccccttcctggtcggattacactcctgcttccagacgacaagtcggttgttcctggtcattgagtacgtcaacggcggggacctgatgttccacatgcagaggcagaggaagctccctgaggagcacgccaggttctacgcggccgagatctgcatcgccctcaacttcctgcacgagagggggatcatctacagggacctgaagctggacaacgtcctcctggatgcggacgggcacatcaagctcacagactacggcatgtgcaaggaaggcctgggccctggtgacacaacgagcactttctgcggaaccccgaattacatcgcccccgaaatcctgcggggagaggagtacgggttcagcgtggactggtgggcgctgggagtcctcatgtttgagatgatggccgggcgctccccgttcgacatcatcaccgacaacccggacatgaacacagaggactaccttttccaagtgatcctggagaagcccatccggatcccccggttcctgtccgtcaaagcctcccatgttttaaaaggatttttaaataaggaccccaaagagaggctcggctgccggccacagactggattttctgacatcaagtcccacgcgttcttccgcagcatagactgggacttgctggagaagaagcaggcgctccctccattccagccacagatcacagacgactacggtctggacaactttgacacacagttcaccagcgagcccgtgcagctgaccccagacgatgaggatgccataaagaggatcgaccagtcagagttcgaaggctttgagtatatcaacccattattgctgtccaccgaggagtcggtgtgaggccgcgtgcgtctctgtcgtggacacgcgtgattgaccctttaactgtatccttaaccaccgcatatgcatgccaggctgggcacggctccgagggcggccagggacagacgcttgcgccgagaccgcagagggaagcgtcagcgggcgctgctgggagcagaacagtccctcacacctggcccggcaggcagcttcgtgctggaggaacttgctgctgtgcctgcgtcgcggcggatccgcggggaccctgccgagggggctgtcatgcggtttccaaggtgcacattttccacggaaacagaactcgatgcactgacctgctccgccaggaaagtgagcgtgtagcgtcctgaggaataaaatgttccgatgaaaaaaaaaa

SEQ ID NO: 5 - protein, 409 aa, homo sapiens
  misc_feature (27)..(27): The ′Xaa′ at location 27 stands for Asp, or Asn.
MDSVMPSQEPPVDDKNEDADLPSEETXGIAYISSSRKHDSIKDDSEDLKPVIDGMDGIKISQGLGLQDFDLIRVIGRGSYAKVLLVRLKKNDQIYAMKVVKKELVHDDEDIDWVQTEKHVFEQASSNPFLVGLHSCFQTTSRLFLVIEYVNGGDLMFHMQRQRKLPEEHARFYAAEICIALNFLHERGIIYRDLKLDNVLLDADGHIKLTDYGMCKEGLGPGDTTSTFCGTPNYIAPEILRGEEYGFSVDWWALGVLMFEMMAGRSPFDIITDNPDMNTEDYLFQVILEKPIRIPRFLSVKASHVLKGFLNKDPKERLGCRPQTGFSDIKSHAFFRSIDWDLLEKKQALPPFQPQITDDYGLDNFDTQFTSEPVQLTPDDEDAIKRIDQSEFEGFEYINPLLLSTEESV

(19) United States
(12) Patent Application Publication
(10) Pub. No.: US 2010/0311663 A1
(43) Pub. Date: Dec. 9, 2010

(54) MEMORY INFLUENCING PROTEIN

(75) Inventor: Todd C. Sacktor, Yonkers, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: THE RESEARCH FOUNDATION OF STATE UNIVERSITY OF NY, ALBANY, NY (US)

(21) Appl. No.: 12/629,359

(22) Filed: Dec. 2, 2009

Related U.S. Application Data
(62) Division of application No. 11/893,723, filed on Aug. 16, 2007, now abandoned.
Application No. 11/893,723 is a continuation-in-part of application No. 09/839,073, filed on Apr. 20, 2001.
(60) Provisional application No. 60/198,802, filed on Apr. 20, 2000.

(51) Int. Cl. A61K 38/10; A61K 31/4355; A61P 25/00; A61P 25/30; A61P 25/32

(52) U.S. Cl. 514/17.7; 514/280

(57) ABSTRACT

The present invention provides methods and compositions for enhancing and/or impairing memory in animals, including humans by the administration of an effective amount of an atypical form of protein kinase C such as protein kinase M zeta (PKMζ) and/or a PKMζ, inhibitor.

### CROSS REFERENCE TO RELATED APPLICATION

This application is a divisional of U.S. patent application Ser. No. 11/893,723 filed on Aug. 16, 2007, which is a continuation-in-part of U.S. Ser. No. 09/839,073, filed on Apr. 20, 2001, which claims the benefit of U.S. Provisional Application No. 60/198,802, filed Apr. 20, 2000.

### FIELD OF THE INVENTION

The present invention provides methods for impairing memory in animals, including humans with the administration of an effective amount of an atypical protein kinase C, including protein kinase M zeta (PKMζ) inhibitor. The present invention is also directed to a method of enhancing spatial, instrumental and classically-conditioned components of long-term memory with an effective amount of PKMζ. The present invention further provides a method of decreasing or attenuating synaptic transmission in an animal suffering from drug or alcohol addiction, post-traumatic disorder, and phobia with the administration of an effective amount of a PKMζ inhibitor.

### BACKGROUND OF THE INVENTION

A common working hypothesis for the physiological basis of memory is that persistent changes in behavior are mediated by long-term modifications in the strength of synapses (Kandel et al. (1982) Science 218:433-443; Bliss et al. (1993) Nature 361: 31-39). The molecular mechanisms for these changes are complex, involving many signal transduction pathways. Overall, however, these mechanisms are divided into two functionally distinct phases: induction, which initiates the long-term modifications, and maintenance, which sustains the changes (Malinow et al (1988) Nature 335:820-824; Schwartz, J. H. (1993) PNAS 90:8310-8313; Schwartz et al (1987) Ann. Rev. Neurosci. 10:459-476). Much of the work to examine these signaling pathways has come from the study of the response to high-frequency afferent stimulation of synapses that causes a long-term increase in synaptic transmission, long-term potentiation (LTP)(Bliss et al. (1993), supra.; Bliss et al. (1973) J. Physiol. 232:331-356; Nicoll et al. (1988) 1:97-103). The vast majority of signaling molecules implicated in LTP affect only induction, but not maintenance. The exceptions are agents that inhibit the catalytic domain of protein kinases, specifically protein kinase C (PKC), which are able both to block LTP induction and reverse its maintenance. (Nishizuka, Y (1988) Nature 334:661-665; Schwartz, J. H. (1993) supra; Schwartz et al (1987) supra.

These two phases can be distinguished experimentally by the timing of the application of pharmacological agents that inhibit signal transduction pathways. When agents are applied prior to a tetanic afferent stimulation and prevent the formation of long-lasting changes, they block induction. If they are applied after the tetanus—and reverse the potentiation that has been established—they affect maintenance.

Several principles have been proposed to characterize mechanisms that might maintain long-term changes in synaptic transmission. First, protein kinases, such as PKC, which transiently enhance synaptic transmission when second messengers are activated, can extend their action by becoming constitutively active kinases that are independent of second messengers. (Schwartz et al (1987) supra; Klann et al. (1991) J. Biol. Chem. 266:24253-24256)

Second, long-term forms of synaptic plasticity are thought to depend upon new protein synthesis, although the critical, newly synthesized molecules that cause synaptic potentiation have not been identified. Stanton et al. (1984) J. Neurosci. 4:3080-3088; Frey et al (1988) Brain Res. 452:57-65; Otani et al (1989) Neurosci. 28: 519-526; Abel et al. (1998) Science 279: 338-341. A similar requirement for new protein synthesis has been observed for long-term memory. Davis et al. (1984) Psychol Bull. 96:518-559; Thompson, R. F. Science 233:941-947; Montarola et al. (1986) Science 234:1249-1254.

While usually considered properties of separate mechanisms, it has been determined that one isoform of PKC possesses both of these features: it is persistently increased during LTP as a constitutively active enzyme, and it is generated by new protein synthesis. Sacktor et al. (1993) Proc. Natl. Acad. Sci. (USA) 90:8342-8346. This form of PKC is PKMζ, the independent catalytic domain of the PKC isoform, which, lacking PKC's autoinhibitory regulatory domain, is autonomously active. Schwartz, J. H. (1993) supra; Sacktor et al. (1993) supra.

PKM is usually thought to be produced by limited proteolysis of PKC, separating the enzyme's regulatory and catalytic domains. This may occur early after a high-frequency tetanus. Some evidence shows, however, that the long-lasting PKMζ may also be derived from a brain-specific mRNA that encodes only the catalytic domain of ζ. Andrea et al. (1995) Biochem. J. 310:835-843; Powell et al. (1994) Cell Growth Differ. 5:143-149.

PKC is a family of multifunctional protein kinases, first discovered by Nishizuka in 1977. Takai et al. (1977) J. Biol. Chem. 252:7603-7609; Inoue et al. (1977) J. Biol. Chem. 252:7610-7616. PKC consists of two domains separated by a hinge region: an amino-terminal regulatory domain, which contains an autoinhibitory pseudosubstrate sequence and second messenger/lipid binding sites, and a carboxy-terminal catalytic kinase domain. PKC is held in an inactive state in the cytosol by the interaction between the regulatory and catalytic domains. When there is an increase in lipid second messengers (or, for some isoforms, Ca²⁺), PKC translocates from the cytosolic to membranous (or cytoskeletal) compartments, and a change in its conformation occurs, displacing the regulatory from the catalytic domain, releasing the autoinhibition, and activating the enzyme. The 10 different forms of PKC are divided into 3 groups: conventional (α, βI, βII, γ), novel (or new, δ, ε, η~, θ), and atypical (ζ, ι/λ), each of which is activated by a distinct set of second messengers. (PKD or PKCμ is a PKC-related molecule with a catalytic domain closer to CaM-kinase). The conventional PKCs are activated by Ca²⁺ and diacylglycerol (DAG); the novel by DAG, but not Ca²⁺; and the atypical by neither DAG or Ca²⁺, but by alternate lipid-second messengers, including arachidonic acid, phosphoinositide 3-OH kinase products, and ceramide.

A second mechanism for permanently activating PKC, also discovered by Nishizuka, is the cleavage by calpain or their proteases at the hinge region, to permanently separate the regulatory from the catalytic domains. The independently active kinase domain is called PKM. (“M” stands for Me, although this requirement turned out to be for the Mg²⁺ in Mg²⁺-ATP). PKM formation results in a persistently active kinase and is not the typical way PKC is activated. It has been found that stable PKM formation occurs endogenously only for a single isoform, and only in brain. Naik et al. (submitted for publication). PKMζ has also been reported in a neuronally differentiated cell line. Oehrlein et al. (1998) Eur. J. Cell. Bio. 77:323-337.

Stable PKM forms for the other isoforms have been observed only in pathological conditions: PKMε, in breast cancer tumor cells (Baxter et al. (1992) J. Biol. Chem. 267: 1910-1917) and heart ischemia (Urthaler et al. (1997) Cardiovasc. Res. 35:60-67) and PKMδ, in apoptosis (Emoto et al. (1996) Blood 97:1990-1996; Denning et al. (1998) J. Biol Chem. 273:29995-30002). Protein kinase M zeta (PKMζ) is a form of protein kinase C which has a fundamental role in the formation and maintenance of memory. PKMζ is a critical molecule in the most widely-studied physiological model of memory, long-term potentiation (LTP) of synapses (Sacktor, et al., (1993) supra.; Osten, et al., (1996) J. Neurosci. 16(8):2444-2451; Hrabetova and Sacktor, (1996) J. Neurosci. 16(17):4324-5333).

It has recently been reported that persistent phosphorylation by PKMζ is required for maintenance of long-term potentiation (LTP) in the hippocampus, as well as for sustaining hippocampus-dependent spatial memory (Pastalkova et al., Science, 313, 1141 (2006)).

It is the neocortex, however, which is believed to ultimately store long-term memory in the mammalian brain (L. R. Squire, P. J. Bayley, Curr. Opin. Neurobiol. 17, 1 (2007).), although opinions differ on whether hippocampus-dependent memories continue to reside in the hippocampus, as well, once they establish themselves in cortex (L. R. Squire, et al (2007) and Nadel, et al., Curr. Opin. Neurobiol. 7, 217 (1997).

Moreover, site-specific brain ablation and inactivation studies have identified the existence of multiple memory systems, each specialized to store a particular type of information in long-term memory (McDonald and White, 1993). These studies however cannot distinguish between a region's role in information storage and its role in information processing because the storage of information in long-term memory appears to be distinct from the processing of information in short-term memory and the transfer of information to or from a different brain region. This distinction between storage and processing was recently made explicit by identifying a molecular mechanism that both maintains late-phase long-term potentiation (late-LTP) of excitatory synaptic transmission and specifically stores information in long-term memory without affecting the acquisition of information in short-term memory (Pastalkova et al., Science 313: 1141 (2006). What is needed therefore is a method and composition which can effect alterations in the brains of mammals which can result in memory enhancements on the one hand and memory disruptions or erasures on the other.

### SUMMARY OF THE INVENTION

The present invention provides methods and compositions for influencing memory in animals, including humans. In one aspect the present invention is directed to enhancing memory including but not limited to spatial, instrumental and classically-conditioned components of long-term memory by the administration of a therapeutically effective amount of one or more atypical forms of protein kinase C (PKC) such as PKMζ.

In another aspect, the present invention is directed to methods of impairing memory in animals.

In another aspect, the present invention provides a method of decreasing or attenuating synaptic transmission in an animal suffering from psychiatric disorders including but not limited to drug or alcohol addiction, post-traumatic disorder and phobia and neurological disorders including but not limited to movement disorders such as dystonia, and restless leg syndrome, comprising the administration of a therapeutically effective amount of one or more atypical forms of PKC such as a PKMζ inhibitor.

In another aspect, the present invention provides a method of impairing or erasing memory in an animal comprising the administration of a therapeutically effective amount of one or more atypical forms of PKC such as a PKMζ inhibitor.

In still another aspect, the present invention provides a method for decreasing or attenuating synaptic transmission or inducing an inability to remember comprising the administration of DNA encoding the human (or animal) sequence of PKMζ inhibitor.

In yet another aspect, the present invention provides a method for decreasing synaptic transmission, comprising the administration of a therapeutically effective amount of a PKMζ inhibitor. Uses for decreasing synaptic transmission include, for example, the treatment of psychiatric disorders including, but not limited to, drug or alcohol addiction, post-traumatic disorder, such as post-traumatic stress disorder, phobias, such as neophobia and neurological disorders including movement disorders such as, but not limited to dystonia and restless leg syndrome.

In still another aspect, the present invention provides a pharmaceutical composition comprising PKMζ or a PKMζ inhibitor and a pharmaceutically acceptable carrier.

### BRIEF DESCRIPTION OF THE DRAWINGS

FIG. 1. Effects of ZIP injections in spatial memory tasks. (FIG. 1A) Performance of the 4-arms baited, 4-arms unbaited 8-arm radial maze task. Learning across 6 days (10 trials per day) was followed by a single retention trial after a 24-hour interval. Two hours prior to the retention trial, each rat received a bilateral intrahippocampal injection of either saline (SAL, n=5-10), ZIP (n=5-10), or an inactive, scrambled amino-acid sequence of the ZIP peptide (Scr-ZIP, n=5-10). The ZIP injection impaired overall performance by increasing reference memory errors. (FIG. 1A) Overall, (FIG. 1B) reference memory and (FIG. 1C) working memory performance. Performance on the standard water maze task during (FIG. 1D) 6 days of training (two 4-trial blocks per day) and (FIG. 1E-G) during the unreinforced-swim retention test after a 24-hour interval. Each rat received a bilateral intrahippocampal injection of saline (n=7), Scr-ZIP (n=7), or ZIP (n=10) two hours before the retention test. (FIG. 1E) Percent time in the target quadrant (FIG. 1F) number of times the position of the escape platform was crossed, and (FIG. 1G) the color-coded time-in-location map for each treatment group during the retention trial. The same blue-to-red scale is used for each map, where the minimum time in the peak, red category is 0.9 sec. ZIP impaired retention of spatial accuracy (F_{2,21}=3.96; P=0.03) but not the spatial search procedure (F_{2,21}=2.08; P=0.15). *P<0.05 ZIP relative to SAL and Scr-ZIP.

FIG. 2. Effects of ZIP injections in conditioned-fear memory tasks. (FIG. 2A) Retention of context-conditioned fear 26-hours after bilateral intrahippocampal injection of saline (SAL, n=4), inactive (Scr-ZIP, n=7) or active ZIP (n=6). ZIP did not impair retention of contextual fear (F_{2,14}=0.15; P=0.86). (FIG. 2B) Retention of tone-conditioned fear after 22-hr post-training bilateral intra-amygdala injections. Retention was tested 2-hr (SAL n=6; Scr-ZIP n=3, ZIP n=10) or 24-hr (SAL n=5; Scr-ZIP n=4, ZIP n=8) after the injection. ZIP impaired retention of tone-conditioned fear (F_{2,33}=4.93; P=0.01). (FIG. 2C) Unconditioned expression of fear after bilateral intra-amygdala injections. Fear was tested 5 min (SAL n=4; ZIP n=4) or 120 min (SAL n=5; ZIP n=5) after the injections. ZIP did not affect the expression of unconditioned fear (F_{1,16}=0.58; P=0.46). (FIG. 2D) Latency to enter the dark compartment during acquisition and retention of inhibitory avoidance. Retention was tested 24-hr after acquisition, two hours after the bilateral intra-amygdala injections (SAL n=5-10; Scr-ZIP n=7, ZIP n=8). ZIP impaired retention of IA. *P<0.05 ZIP relative to SAL and Scr-ZIP.

FIG. 3. Erasure of long-term CTA memory by a single application of the PKMζ inhibitor ZIP into the insular cortex (IC). FIG. 3A. ZIP was microinfused bilaterally into the IC 3 days after CTA training, and memory was tested 1 week or 1 month later. CTA memory was blocked at both time points, as compared to control rats microinfused into the IC with the vehicle only and tested 1 month later. The dashed line indicates equal preference for the CS and water in the test, i.e., aversion index (AI)=50 (see Methods). AI of <50 indicates preference for the CS, which may develop over time for saccharin and some other CSs in naive or CTA-extinguished rats, but AI doesn't decline below 20-30 even in naive rats. The AI of the ZIP groups hence reflects massive loss of memory. For statistics see text. FIG. 3B. ZIP was microinfused bilaterally into the IC at the indicated times after training. Again, this led to massive decline in memory tested 2 hours (3 days time-point) or one day (7 and 25 days time-point) later. FIG. 3C. The effect of ZIP on long-term CTA memory remains even after reactivation, and there is no evidence for spontaneous recovery or recovery after US-reinstatement. Rats were trained on CTA and tested once 3 days later, followed immediately by bilateral microinfusion of ZIP into the IC. This led to decline of the memory tested a day later. Further testing unveiled neither spontaneous recovery, which took place in the no-test interval between days 4 and 12 in the control group, nor UCS-reinstatement (LiCl, day 13).

FIG. 4. The PKMζ inhibitor has no effect on memory of familiarity in the IC. FIG. 4A. In latent inhibition (LI), preexposure to the tastant attenuates the potency of that tastant to serve as CS in subsequent CTA training (compare Vehicle, LI training to No LI training). LI of CTA can serve to quantify taste familiarity. Microinfusion of ZIP into the IC after the exposure to the tastant in the LI protocol (ZIP, LI training) had no effect on familiarity. FIG. 4B. Encounter with an unfamiliar tastant provokes neophobia, which declines over repeated non-reinforced exposures to that same tastant (days 1-6). Attenuation of neophobia is another protocol to quantify familiarity. Application of ZIP into the IC had no effect on familiarity in this protocol, as well.

FIG. 5. PKMζ inhibition in the hippocampus does not erase CTA memory. ZIP was microinfused bilaterally into the hippocampus 3 days after CTA training and memory was tested starting a day later. These data also demonstrate that the effect of ZIP on CTA memory in the IC is region-specific.

FIG. 6 shows the protein (SEQ ID NO: 5) and coding DNA sequence human PKM (SEQ ID NO: 3). The entire DNA sequence of SEQ ID NO: 4 is also shown.

### DETAILED DESCRIPTION OF THE INVENTION

The present invention provides methods and compositions for influencing memory. In one embodiment, the present invention provides compositions and methods for enhancing memory, including but not limited to spatial, instrumental and classically-conditioned components of long-term memory by the administration of a therapeutically effective amount of one or more atypical forms of PKC. In a preferred embodiment an atypical form of PKC is PKMζ. In accordance with the present invention it has been determined that PKMζ is both necessary and sufficient for the long-term maintenance of LTP. Moreover, it has been determined in accordance with the present teachings that the function of PKMζ is to store and consolidate memories in the brain. Furthermore, it has been discovered in accordance with the present invention that long-term associative memory in the cortex can be disrupted or erased with administration of PKMζ inhibitor peptides of the present invention. By “PKMζ inhibitor peptides” and “PKMζ inhibitor” is meant chelerythrine and myristolated zeta inhibitory pseudosubstrate peptide (MZIP).

In accordance with the present invention, members of the class of compounds known as atypical forms of PKC such as protein kinase M zeta (PKMζ) have been found to maintain or consolidate long term changes in synaptic strength in vertebrates, the mechanism for long term memory. The present invention elucidates PKMζ's role in maintaining enhanced synaptic transmission with studies of long-term potentiation (LTP). Conversely, inhibition of PKMζ may permit erasure of unpleasant memories or disruption of memory, which may be useful in the treatment of traumatic stress disorders, phobias and acute or chronic pain, as well as drug and alcohol addiction.

Other agents that have been proposed to enhance memory are essentially stimulants (like coffee) or agents designed to enhance the induction of long-term potentiation (LTP)-like processes (such as drugs to increase cAMP). PKMζ is the first molecule whose function is to maintain or enhance memories in vertebrates. In accordance with the present invention, when PKMζ is injected into neurons it persistently enhances synaptic transmission.

In one embodiment the present invention contemplates a method of enhancing memory, such as spatial, instrumental and classically-conditioned components of long-term memory in an animal comprising the administration of a therapeutically effective amount of one or more atypical forms of PKC such as PKMζ, or DNA encoding PKMζ message. By “therapeutically effective amount” is meant an amount of an atypical form of PKC high enough to significantly positively modify the condition to be treated but low enough to avoid serious side effects (at a reasonable benefit/risk ratio), within the scope of sound medical judgment.

In still another embodiment, the present invention contemplates a method of decreasing, disrupting or attenuating synaptic transmission in an animal suffering from a psychiatric disorder including, drug and/or alcohol addiction, post-traumatic disorder and phobia comprising the administration of a therapeutically effective amount of one or more PKMζ inhibitors. By “therapeutically effective amount”, as related to PKMζ inhibitors is meant an amount of at least one PKMζ inhibitor high enough to disrupt, decrease, attenuate or erase memories in mammalian subjects while avoiding serious side effects (at a reasonable benefit/risk ratio), within the scope of sound medical judgment.

The present invention also contemplates a method of impairing the ability to remember or erasing memory in an animal by the administration of a therapeutically effective amount of a PKMζ inhibitor. In preferred embodiments the PKMζ inhibitor is chelerythrine. In another preferred embodiment the PKMζ inhibitor is myristolated zeta inhibitory pseudosubstrate (MZIP) peptide (myr-Ser-Ile-Tyr-Arg-Arg-Gly-Ala-Arg-Arg-Trp-Arg-Lys-Leu-OH (SEQ ID NO:1)), or a dominant negative form of PKMζ such as, for example, PKMζ-K281W, or antisense to PKMζ mRNA. MZIP has an IC50 of 10-100 nM for PKMζ and 10,000 nM for PKC gamma and therefore is a more specific inhibitor than chelerythrine. Candidates for the induction of memory disruption or memory erasure contemplated by the present invention are preferably humans having, for example, post-traumatic disorders, phobias and drug and/or alcohol addiction.

The present invention also contemplates a method of reducing or decreasing synaptic transmission in selective areas of the brain including the cortex by the local or systemic administration of a therapeutically effective amount of PKMζ inhibitor. Candidates for the reduction of synaptic transmission contemplated by the invention are preferably humans having, for example, disorders of pain, drug and/or alcohol addiction, post-traumatic disorder and phobias, such as neophobia, for example.

Still another embodiment of the present invention contemplates pharmaceutical compositions containing one or more atypical forms of PKC such as, for example, PKMζ.

Yet another embodiment of the present invention contemplates pharmaceutical compositions containing one or more PKMζ inhibitors.

The active ingredients of a pharmaceutical composition containing PKMζ or a nucleic acid encoding PKM is contemplated to exhibit effective therapeutic activity, for example, in enhancing memory, and treating brain and spinal cord injuries. Thus the active ingredients of the therapeutic compositions containing PKMζ are administered in therapeutic amounts which depend on the particular memory to be enhanced. For example, final concentrations of PKMζ in mammalian brain to be achieved by administration, can be about 1 nanomolar. The dosage regimen can be adjusted to provide the optimum therapeutic response. For example, several divided doses can be administered daily or the dose can be proportionally reduced as indicated by the exigencies of the therapeutic situation. Administration of one or more atypical forms of PKC such as, for example, PKMζ into the brain or spinal cord can be intracranially or intrathecally, i.e., by intrathecal pump or repository. Depending on the route of administration, the active ingredients which comprise PKMζ can be required to be coated in a material to protect said ingredients from the action of acids and other natural conditions which may inactivate said ingredients.

For example, PKMζ can be administered in an adjuvant or in liposomes. Adjuvants contemplated herein include resorcinols, nonionic surfactants such as polyoxyethylene oleyl ether and n-hexadecyl polyethylene ether. Liposomes include water-in-oil-in-water P40 emulsions as well as conventional liposomes.

Under ordinary conditions of storage and use, the preparations of the present invention contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yield a powder of the active ingredient plus any additional desired ingredient from previously sterile-filtered solution thereof.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of the active material calculated to produce the desired therapeutic effect in association with the required pharmaceutical carrier. The specification for the novel dosage unit forms of the invention are dictated by and directly depending on (a) the unique characteristics of the active material and the particular therapeutic effect to be achieved, and (b) the limitations inherent in the art of compounding such as active material for the treatment of injury in living subjects having a condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, result in achieving, for example, about 0.1 to about 10 nanomolar concentrations of PKMζ in the brain or spinal cord.

As used herein “pharmaceutically acceptable carrier” includes any and all solvents, dispersion media, coatings, antibacterial and antifungal agents, isotonic and adsorption delaying agents, and the like. The use of such media agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

Administration of an atypical form of PKC, such as PKMζ can also include altered forms or derivatives of PKMζ or drugs that enhance its activity, stability, or accessibility to the nervous system. The identification of applicable PKMζ enhancing drugs are readily tested or screened by examining the effects of drugs or PKMζ's phosphorylation in vitro or on PKMζ's effect on synaptic transmission when injected into neurons.

Administration of PKMζ DNA into brain or spinal cord can also be by gene-transfer technology. Such technologies include, but are not limited to, viruses, liposomes, and altered forms or derivatives of DNA or RNA.

Administration of inhibitors of PKMζ activity include drugs, such as chelerythrine, myristolated zeta inhibitory pseudosubstrate peptide and altered forms of PKMζ that, through dominant negative effects inhibit endogenous PKMζ's activity or effectiveness. Such dominant negative agents include, but are not limited to, inactive forms or portions of PKMζ. Inhibition of PKMζ function can also include decreasing levels of endogenous PKMζ through administration of antisense or RNAi to the sequence of PICK. The active ingredients of a pharmaceutical composition containing a PKMζ inhibitor is contemplated to exhibit effective therapeutic activity, for example, in erasing memory. Thus the active ingredients of the therapeutic compositions containing PKMζ inhibitors of the present invention are administered in therapeutic amounts which depend on the particular memory to be erased. For example, final concentrations of PKMζ inhibitor in brain to be achieved by administration may be about 1 to 5 micromolar. The dosage regimen can be adjusted to provide the optimum therapeutic response within sound medical judgment of the skilled artisan. For example, several divided doses may be administered daily or the dose can be proportionally reduced as indicated by the exigencies of the therapeutic situation. Administration of one or more PKMζ inhibitors into the brain or spinal cord may be intracranially or intrathecally, i.e., by intrathecal pump, injection or repository. Depending on the route of administration, the active ingredients which comprise PKMζ inhibitors may be required to be coated in a material to protect said ingredients from the action of acids and other natural conditions which may inactivate said ingredients.

For example, PKMζ inhibitors can be administered in saline, an adjuvant or in liposomes. Adjuvants contemplated herein include resorcinols, non-ionic surfactants such as polyoxyethylene oleyl ether and n-hexadecyl polyethylene ether. Liposomes include water-in-oil-in-water P40 emulsions as well as conventional liposomes.

Under ordinary conditions of storage and use, the preparations of the present invention contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yield a powder of the active ingredient plus any additional desired ingredient from previously sterile-filtered solution thereof.

It is especially advantageous to formulate compositions in dosage unit form for ease of administration and uniformity of dosage. “Dosage unit form” as used herein refers to physically discrete units suited as unitary dosages for the mammalian subjects to be treated; each unit containing a predetermined quantity of the active material calculated to produce the desired therapeutic effect in association with the required pharmaceutical carrier. The specification for the novel dosage unit forms of the invention are dictated by and directly depending on (a) the unique characteristics of the active material and the particular therapeutic effect to be achieved, and (b) the limitations inherent in the art of compounding such as active material for the treatment of injury in living subjects having a condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, result in achieving, for example, about 0.1 to about 10 micromolar concentrations of PKMζ inhibitor in the brain or spinal cord. In one embodiment, a PKMzeta inhibitor is injected into the brain of the subject such that a final concentration in the area of the brain is about 1-10 micromolar and preferably about 5 micromolar. In another embodiment, 10 nanoMoles of PKM zeta inhibitor in 1 microliter bolus injection will spread to an area of the brain approximating 3 mm-5 mm.

The following Examples serve to further illustrate the invention without in any way limiting same.

### Example 1

### Spatial, Instrumental and Classically-Conditioned Components of Long-Term Memory are Maintained By PKMzeta (PKMζ)

A persistently active kinase, PKMzeta (PKMζ), is both necessary and sufficient for maintaining late-LTP in the hippocampal slice (Ling et al., Nature Neuro. 5:295-296 2002). This mechanism of late-LTP maintenance was reversed in vivo by injecting a PKMζ inhibitory peptide (MZIP) directly into the hippocampus. MZIP, a potent and selective inhibitor of PKMζ returned potentiated synaptic transmission to baseline levels at widespread hippocampal locations a day after the potentiating stimulation, yet had no effect on non-potentiated, baseline synaptic transmission. The same injection of MZIP erased 1 day-old and 1 month-old long-term memory that had been acquired in a place avoidance task. Injecting MZIP into the insular cortex also selectively erased 3 day-old and 25-day old long-term taste aversion memory.

Despite erasing stored information, MZIP had no effect on information processing. Injecting MZIP into the hippocampal and neocortical sites spared short-term recognition memory. These findings established that MZIP selectively erases information from late-LTP-dependent long-term memory without affecting the ability to acquire and express new information. The ability of site-specific MZIP injections to selectively erase information from long-term memory makes this a powerful new tool for specifying the type of information that is stored in a brain region by late-LTP-dependent long-term memory, distinct from the region's role in processing information that is stored elsewhere.

The present invention therefore used post-training intracranial injections of MZIP to determine whether local information storage in the hippocampus accounts for its role in spatial memory, and whether local storage in the amygdala accounts for its role in fear-motivated memory. The results provide resolutions to three long-standing controversies in memory research, and furthermore, they indicate that the role of the hippocampus and amygdala in long-term information storage is specific to the demands of the individual task and, relative to the conclusions of ablation studies, substantially more restricted than previously thought.

### Spatial Memory

Spatial reference memory is distinguished from spatial working memory in the 8-arm radial maze because information about which arm locations are consistently baited is valid across trials, whereas working memory requires spatial information for which arm locations were visited within a trial, information that is valid only for the specific trial. Lesions of the hippocampus increase working memory errors but not reference memory errors (Olton et al., Brain Behav. Science 2: 313-365 1979). Results from this research continue to be perplexing, however because a large number of lesion studies has established the idea that the hippocampus is critical for spatial reference memory in the water maze task and other tests of spatial reference memory (Morris et al., Nature 297: 681-683 1982).

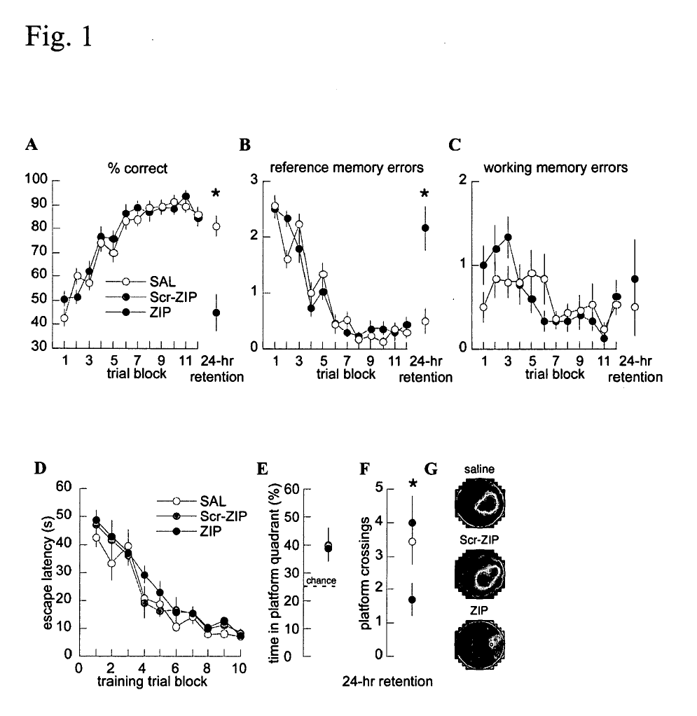

Rats learned the 4-baited, 4-unbaited 8-arm radial maze task and after 30 trials (3 days) performance was asymptotic and optimal for an additional 30 trials (days 4-6; FIG. 1A). On day 7, two hours before training, bilateral intrahippocampal injections of saline or Scr-ZIP (Scrambled ZIP) did not alter performance during testing that began two hours later. In contrast, injections of ZIP caused performance to drop to the level of naïve rats (FIG. 1A). This indicated a specific loss of spatial reference memory because the ZIP-injected rats made many reference memory errors (FIG. 1B) without making more working memory errors than the control rats (FIG. 1C). This, along with other features of performance following ZIP injection indicated that memory for the procedural aspects of the task were unaffected. Thus PKMζ activity in the dorsal hippocampus stores information for spatial reference memory but not the spatial and procedural information needed for working memory in this standard 8-arm radial maze task.

The inventor also examined whether spatial reference memory in the standard water maze escape task is also stored by maintained PKMζ activity in the hippocampus. Whether the hippocampus is crucial for spatial reference memory in the water maze is now controversial, especially following the recent use of tetanic stimulation to saturate synaptic transmission and pharmacological blockade of NMDA receptors to prevent late-LTP. Although these procedures, along with permanent and functional lesions of dorsal hippocampus, impair learning and memory of the escape location the impairment is absent in rats that learned the water maze procedure but not the particular escape location prior to the amnestic intervention.

The invention employed a spatial training protocol that establishes a hippocampus-dependent memory, which on day 6 can be demonstrated by an inability to localize searching for the platform on a probe trial following hippocampal inactivation (Kubik and Fenton, J. Neurosci. 25: 9205-9212, 2005). In this experiment, rats learned the location of the escape platform during five days of training (FIG. 1D). Similar to saline-injected rats, the rats repeatedly crossed the platform location and concentrated their search in the correct quadrant if they were injected with Scr-ZIP two hours before the probe test on day 6 (FIG. 1E-G). In contrast, ZIP injections diminished the accuracy of searching but did not impair the search procedure. Compared to the rats injected with saline or the control compound, the ZIP-injected rats crossed the target location fewer times (FIG. 1E; P<0.01) but they concentrated their search in the correct quadrant of the pool just as much as rats that received the control injections (FIG. 1F-G; P>0.05). Thus, it can be concluded that persistent PKMζ activity in dorsal hippocampus stores information necessary for normal spatial accuracy but not the information upon which the spatial search procedure is based.

### Fear-Associated Memory

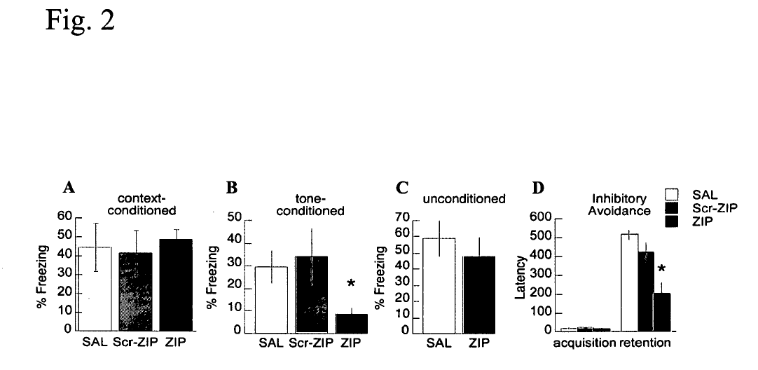

The present invention also investigated whether information for contextual conditioned fear is stored by PKMζ activity in the dorsal hippocampus. Rats were conditioned in a combined context and tone-conditioned fear protocol that long-term retention of the contextual fear response is impaired by post-training lesion of the dorsal hippocampi (Kim and Fanselow, Science 256: 675-677, 1992;). In contrast, ZIP injections into the dorsal hippocampi 22 hours after context-shock pairing failed to alter contextual freezing tested 26 hours later (FIG. 2A; P>0.86). The ZIP also did not impair tone-associated fear tested in a novel chamber 74 hours after the infusions. Additional context-shock pairing protocols produced the same outcome leading to the conclusion that information for contextual fear memory expression is not stored by PKMζ-dependent late-LTP in the hippocampus.

The invention then examined tone-associated fear memory. According to a “storage” hypothesis, a network of structures centered on the basal and lateral nuclei of the amygdala (BLA) may be the locus of storage for the memory. The inventors tested whether persistent PKMζ activity in the BLA maintains the information that is needed to retain tone-associated fear. Rats received a single tone-shock pairing trial and 22 hours later they were injected with saline, Scr-ZIP, ZIP. The saline- and Scr-ZIP-treated rats expressed normal conditioned fear 2 hours and 24 after the injection, but conditioned freezing was impaired in the ZIP-injected rats at both retention delays. The results of the two retention delays were indistinguishable and therefore analysed together (FIG. 2B; P<0.02).

Ablation of the BLA attenuates freezing to the shock, itself (REFs), which may confound the interpretation of whether information is stored in the BLA or whether late-LTP in the BLA is required for the expression of fear. To address this issue, the invention examined rats that were injected with saline or ZIP and either 5 minutes or two hours later, unconditioned freezing to shock was measured. ZIP did not affect unconditioned freezing at either time point (FIG. 2C; P>0.42). Because ZIP did not alter the ability to express fear, but virtually eliminated conditioned fear, the invention concludes that PKMζ activity in the BLA stores the information that is required for tone-associated fear, while sparing the function of the BLA in expressing fear, itself.

The conclusion that information is stored in the BLA contradicts the “modulation” hypothesis, that the amygdala plays a processing role in conditioned behavior, specifically by modulating the strength of information storage at extra-amygdala sites rather than to store associative information in the BLA (McGaugh et al., PNAS USA 93: 13508-13514, 1996; McGaugh, Annu Rev Neurosci 27: 1-28, 2004). Unlike classically-conditioned fear memories, the modulation hypothesis is based on a large body of work using inhibitory avoidance (IA), a form of instrumental conditioning. Thus the inventors tested whether post-training inhibition of PKMζ activity in the BLA would impair the retention of IA. Injecting ZIP into the BLA 22 hours after IA conditioning impaired retention of the conditioned response that was tested 2 hours after the injection (P<0.05). Consistent with the storage hypothesis, information required for IA was stored in the BLA by persistent PKMζ activity.

### Discussion

Previously, identifying the type of memory that is stored in a brain structure has relied on its ablation or inactivation by anesthetic agents. These methods, however, eliminate all physiological responses emanating from the brain region. This confounds identifying the type of information that is stored in long-term memory in a particular region of the brain because a part of the brain that stores memory may also participate in expressing the conditioned behavior, and in relaying the information to and from other anatomically-related brain regions.

In the 8-arm radial maze task, injecting ZIP into the dorsal hippocampus resulted in the complete loss of information supporting spatial reference memory, but no effect on working memory or the ability to do the win-shift foraging procedure (FIG. 1A). Similarly, the effect of ZIP injection in the standard water maze task was the elimination of information supporting spatial accuracy (FIG. 1E), while information needed for the general place response to search in the platform quadrant of the pool was spared (FIG. 1F-G). In contrast, the equivalent ZIP injection did not impair context-associated fear at all (FIG. 2A). Thus the long-term information encoded within the hippocampus by LTP appears to be required for fine, accurate spatial reckoning or precise discrimination between related memories of location, as between the arms in the radial maze. On the other hand, information encoded by late-LTP in the hippocampus are not required for working memory in the radial arm maze (which might be mediated by transient early-LTP or coarser-grained memories of spatial position or context, which may be encoded elsewhere.

The present invention confirms that the dorsal hippocampus stores the information needed for accurate, discriminative learned spatial responses. Furthermore, the well-documented roles of the dorsal hippocampus in spatial working memory general place responses (Morris et al., 1982), and contextual memory is unlikely to be due to late LTP-mediated, dorsal hippocampal long-term information storage. Because these other roles were observed in ablation studies but not with ZIP injections, the dorsal hippocampus may also function as a relay or a computational structure, providing access to and operations upon coarse spatial information that is either stored at extra-hippocampal sites or by local non-LTP mechanisms.

Similarly, the invention's findings in the BLA separate and distinguish between its role in the storage of fear-motivated information and its role in the expression of this information. ZIP injections in the BLA resulted in a loss of information to support tone-associated fear (FIG. 2B) as well as response-associated fear (FIG. 2D), but not the information needed to express fear itself, in response to an unconditioned shock (FIG. 2C), as observed, for example, with ablations of the BLA (REFs).

The invention thus recognizes that a neural network may have multiple roles in learning and memory: on the one hand, storing information in local synapses, and on the other, relaying information or performing computations on information stored elsewhere. This finding was made possible by the identification of PKMζ as the first specific molecular mechanism for long-term information storage, and the ability to selectively inhibit its activity without altering other aspects of neurotransmission.

### Example 2

### Memory Erasure Studies

Male Wistar rats (60 days old, 250-350 gm) were caged individually at 22±2° C. in a 12 hour light-dark cycle. Water and food were available ad libitum unless otherwise indicated. All experiments were approved by the Weizmann Institute of Science Animal Care and Use Committee.

Chemicals: The PKMζ inhibitor ZIP (myr-SIYRRGARRWRKL-OHSEQ ID NO: 1) was dissolved in phosphate-buffered saline (PBS, the vehicle), to a concentration of 10 nmol/μl. A scrambled peptide (myr-RLYRKRIWRSAGR-OH, SR1(Seq ID NO. 2) in the same concentration or the vehicle were used in the control groups, as indicated in the text. Both peptides were purchased from Quality Controlled Biochemicals, Hopkinton, Mass.

Behavioral procedures: Conditioned taste aversion (CTA) was induced and tested as previously described (Rosenblum et al., Behav. Neural Biol. 59: 49, (1993); Rosenblum et al., J. Neurosci. 17: 5129, (1997)). Briefly, rats were deprived of water for 24 hours, and then trained over 3 days to obtain their daily water ration within 10 min from 2 pipettes, each containing 10 ml of tap water. On day 5, water was replaced with the tastant solution (saccharine 0.1% or glycine 1% w/v, the conditioned stimulus, CS). This was followed 40 min later by an intraperitoneal (i.p.) injection of 0.15M LiCl (the unconditioned stimulus, UCS). Testing was subsequently performed at the times indicated below, by presenting the rats with an array of six pipettes, 3 each with 5 ml the relevant taste and 3 each with 5 ml water. The aversion index (AI) was defined as [(water consumed)/(water+taste consumed)×100].

In the latent inhibition protocol (LI), rats were exposed to the tastant for 10 min in 2×10 ml pipettes once a day for two consecutive days. CTA training with the same tastant as the CS was performed 3 days later, as above. In attenuation of neophobia (AN), rats were given free access to an array of six pipettes, 3 each with 5 ml saccharine 0.5% and 3 each with 5 ml water, for 10 min, once a day for 6 consecutive days, and the tastant consumption monitored daily.

Surgery and Brain Targeted Microinfusions: Rats were anesthetized with 0.4 ml/kg Pental, restrained in a stereotaxic apparatus, and implanted bilaterally with a stainless steel guide cannulae (23 gauge) aimed 1.0 mm above the gustatory neocortex (AP +1.4 mm, L ±5.3 mm, V 5.4 mm relative to Bregma), or above the dorsal hippocampus (AP −3.5 mm, L ±2.6 mm, V 2.6 mm relative to Bregma). The cannulae were positioned in place with acrylic dental cement and secured by two skull screws. A stylus was placed inside the guide cannulae to prevent clogging. Rats were allowed 1 week to recuperate before being subjected to experimental manipulations. For microinfusions, the stylus was removed, and a 28-gauge injection cannula, extending 1.0 mm from the tip of the guide cannula, was inserted. The injection cannula was connected via PE20 tubing to a Hamilton microsyringe driven by a microinfusion pump (CMA/100; Carnegie Medicin). Microinfusions were performed bilaterally in a 1-μl volume per hemisphere delivered over 1 min. The injection cannula was left in position before withdrawal for an additional 1 min to minimize dragging of the injected liquid along the injection tract.

Histology: Following completion of the experimental protocol rats were deeply anaesthetized and 1 μl of India ink was microinfused into the insular cortex. After decapitations the brains were quickly removed, frozen on dry ice and kept in −20° C. Coronal slices (30 μm) were cut in a cryostat, stained with cresyl violet, and analyzed to verify the microinfusion sites.

Statistics: t-test (2-tail unpaired) was used for comparison of two groups. One way ANOVA and repeated measures ANOVA were used for comparisons of more than 2 groups, and in cases of repeated tests, respectively, with a level of 0.05.

### Conditioned Taste Aversion-Memory Erasure

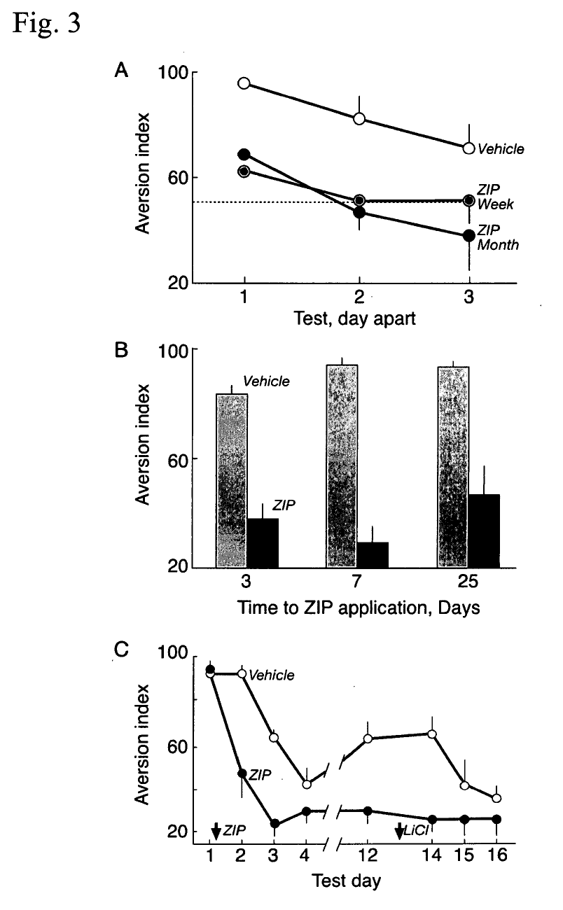

Rats were trained on conditioned taste aversion (CTA) using 0.1% saccharine as the conditioned stimulus (CS), and 3 days later, the inventors microinfused the pseudosubstrate inhibitor ZIP bilaterally into the IC. The control group was microinfused with vehicle only, ZIP-rats were divided into two groups, one group was tested 1 week later and the other 1 month later. As can be seen in FIG. 3A, ZIP in the IC blocked CTA memory tested either 1 week or 1 month later (one-way ANOVA, F(2,16)=7.61, p<0.005). Post-hoc comparisons unveiled no significant difference between the two ZIP groups; however, each is significantly different from the control (p<0.05). The difference persisted on continuous testing in an extinction mode (repeated-measures ANOVA, Group effect, F(2,16)=6.17, p<0.01, Test effect, F(2,32)=8.91, p<0.001). The 1 week and 1 month ZIP groups do not differ from each other, but each is significantly different from control (p<0.05).

In a further experiment, rats were trained on CTA and then microinfused ZIP into the IC at various time points, ranging from 3 to 25 days after training, followed by CTA testing. The PKMζ inhibitor was effective in blocking CTA memory at all the time points tested (FIG. 3B; p<0.001 for the difference between the 3 days and 7 days groups each and control, p<0.005 for the difference between the 25 days group and control). These data also show that the effect of ZIP on long-term memory is rapid (within 2 hrs at most, FIG. 3B, 3 days time point) and is not eliminated by intensifying CTA training (FIG. 3B, 25 days time point).

To exclude the possibility that the effect of the PKMζ inhibitor is unique to the CS used, saccharin was replaced with glycine 1% as the CS. ZIP was microinfused into cortex 3 days after CTA training. Scrambled inactive ZIP was microinfused into the IC as control (1). A memory test one day later unveiled AI=74.7+/−6.5 in the ZIP group, and 98.2+/−1.05 in the scrambled ZIP group (n=8 each, p<0.005). These data show that the effect is not unique to the CS used, and that it is specific to inhibition of PKMζ activity.

ZIP was also tested to assess whether or not it retains the ability to block the memory if administered right after retrieval. When administered into cortex 1-4 min after the first retrieval test, ZIP blocked the long-term memory as well (FIG. 3C; repeated-measures ANOVA for the first two tests, significant Group effect, F(1,12)=13.09, p<0.005, significant Test effect, F(1,12)=17.95, p<0.005, and significant GroupXTests interaction, F(1,12)=17.53, p<0.005)).

To further examine the possibility that the PKMζ inhibitor might block long-term memory performance only transiently, ZIP-treated rats were tested over time, to unveil potential spontaneous recovery, and in addition, after about 2 weeks, the UCS was reapplied, to elicit potential reinstatement of latent memory. None of these manipulations demonstrated any evidence for recovered memory (FIG. 3C). Repeated-measures ANOVA for the tests conducted on the vehicle group on days 4 and 12 shows significant Group effect, F(1,12)=5.79, p<0.05, and a trend towards GroupXTest interaction, F(1,12)=4.29, p=0.06. This indication for spontaneous recovery was clearly missing in the ZIP group. In addition, the non-significant difference between the groups on test 5 (p=0.28), becomes significant on days 12 and 14 (p<0.01), which might be attributed to spontaneous recovery and UCS-reinstating effects in the control group but not in the ZIP group.

A further experiment was conducted to assess whether or not PKMζ inhibition can disrupt more than one association at a time? Rats were trained on CTA to saccharin (CS1), and 2 days later on CTA to glycine (CS2). These two tastants are perceived differently by the rat (Stehberg & Dudai, unpublished). One week later, ZIP was microinfused into the IC, and a day later, a test schedule was initiated in which the rats (n=8) were tested for CTA of CS1 and CS2, consecutively, one day apart over 6 days. Both associations, that of CS1-UCS and of CS2-UCS, were disrupted: AI on the first test for CS1 association was 94+/−3.16 in the control group (n=7), 70.3+/−7.09 in the CS1 group (F (1,13)=8.41, p<0.05). AI on the first test for CS2 association was 97.6+/−0.97 in the control group, 78.9+/−5.8 in the CS2 group (F(1,13)=8.61, p<0.05). No significant difference was detected among the groups in subsequent extinction rate, indicating lack of recovery from the ZIP effect in repetitive testing (repeated-measures ANOVA, GroupXTest interactions, F(2,26)<1, n.s.).

The effect of the PKMζ inhibitor in the IC on the ability to encode, as opposed to retain, CTA long-term memory, in two different ways was also tested. First, ZIP was microinfused into the IC 2 hr before exposure to a glycine CS in CTA training, and tested 3 days later. No effect of ZIP on the acquisition of CTA long-term memory was found (ZIP group, 85.4+/−5.0, n=8, vehicle, 87.4+/−5.9, n=7, one way. The inventors took rats that were trained on CTA to saccharin and then treated with ZIP in the IC to erase the memory (FIG. 3B, 3 days time point), and a week later, subjected them to a new CTA training to glycine. There was no difference between the ZIP and the control rats in their ability to reacquire CTA (ZIP group, 93.2+/−2.3, n=9 vehicle, 95.0+/−2.9, n=5 one-way ANOVA, F(1,12)<1, p=0.62).

Ample data indicate that the IC takes part in, and is obligatory for, the process of detection, encoding and consolidation of taste familiarity. Two taste familiarity paradigms were used to determine the ability of inhibition of PKMζ to disrupt taste familiarity once formed. The first paradigm is latent inhibition of CTA. Since CTA is stronger when the taste CS is unfamiliar, preexposure to the CS in an LI protocol attenuates later CTA training to the same CS, hence the CTA performance can serve as a detector to quantify taste familiarity. Introduction of ZIP into the IC after the incidental exposure to the unfamiliar taste in the LI training protocol, had no effect on LI, indicating lack of erasure of taste familiarity in the IC by ZIP (FIG. 2A; one-way ANOVA for the first test, (F(2,37)=7.77, p<0.005)). Post-hoc comparisons unveils no significant difference between the ZIP-LI and the vehicle-LI groups; however, both these groups are significantly different from the no-LI group (p<0.01). Repeated-measures ANOVA shows significant Group effect (F(2,37)=6.83, p<0.005) and significant Test effect (F(2,74)=15.94, p<0.001). Again, post-hoc comparisons show no significant difference between the two LI groups, and each of these groups is significantly different from the no-LI group (p_{<}0.01).

### Neophobia Inhibiton-Memory Erasure

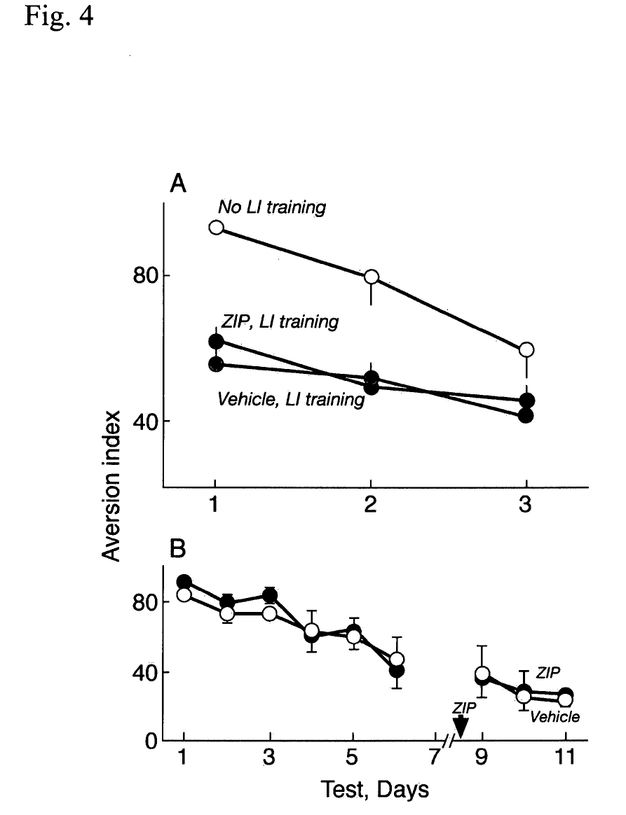

A second familiarity paradigm was also used, attenuation of neophobia (Buresova et al., Behav. Brain Res. 1: 299, (1980)). In this method, the rats are presented with an unfamiliar tastant that invokes a significant fear-of-the-new, and then repeatedly presented with the same tastant. Over time, the neophobia decreases; hence attenuation of neophobia is a measure of familiarity. PKMζ inhibition has no effect on acquired familiarity in this paradigm as well (FIG. 4B, repeated-measures ANOVA unveils significant attenuation of neophobia in the repeating tests, F(8,112)=21.01, p<0.001, however, no significant difference between the groups, F(1,14)<1, p=0.85).

The effect of the PKMζ inhibitor on long-term CTA memory in IC is in line with previous reports that the IC is critical for consolidation, storage, extinction and reconsolidation of CTA (Rosenblum et al., Behav. Neural Biol. 59: 49 (1993); Bermudez-Rattoni, Nature Rev. Neurosci. 5: 209, (2004); Berman et al., Science 291: 2417, (2001); Eisenberg et al., Science 301: 1102, (2003)). Although the map of CS-UCS association sites in CTA encoding is still incomplete and very probably includes subcortical structures once the association is formed, the IC is believed to be likely to store the associative hedonic or incentive value of the conditioned taste (Balleine et al., J. Neurosci. 20: 8954-8964, (2000)).

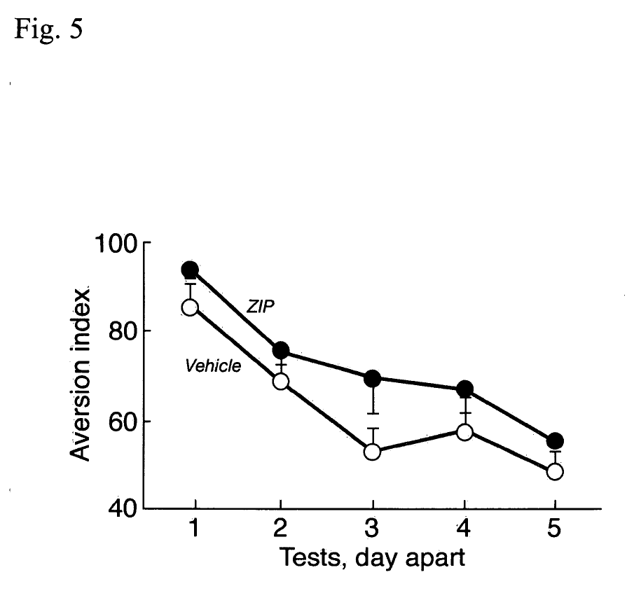

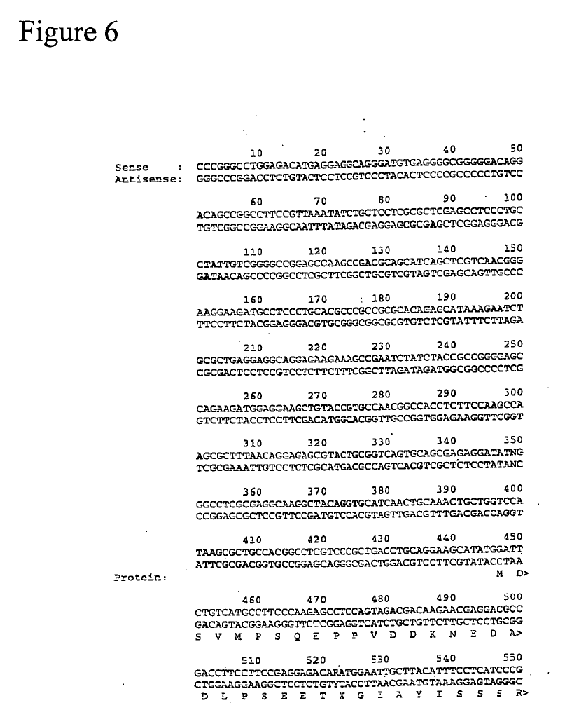

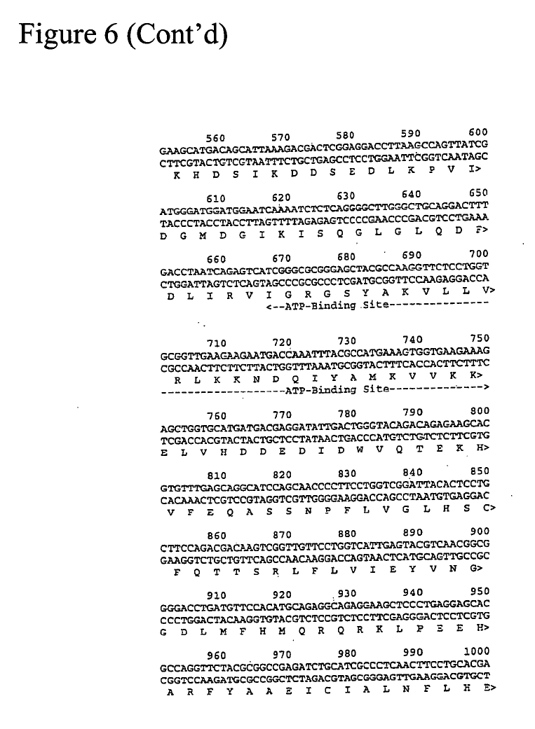

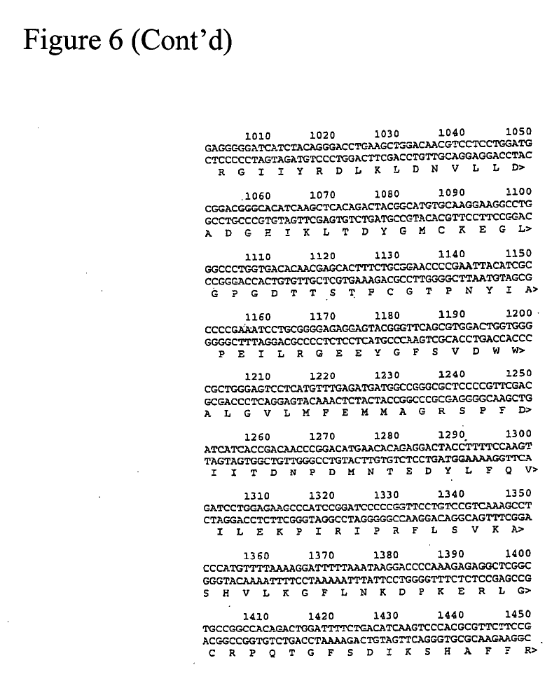

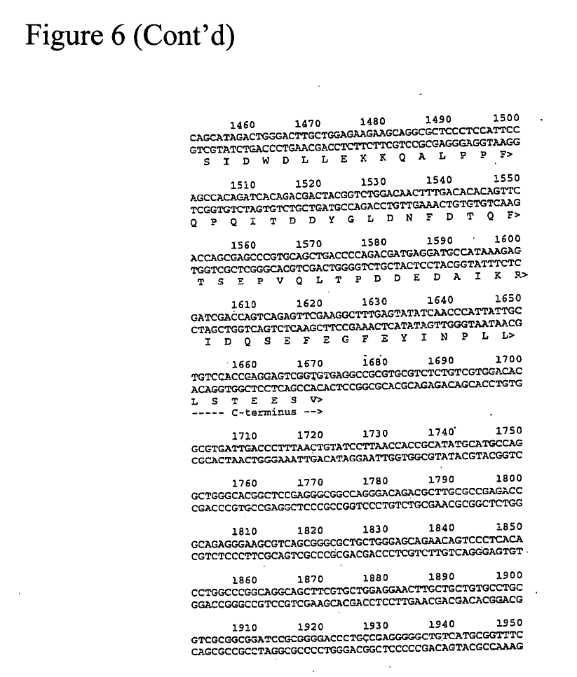

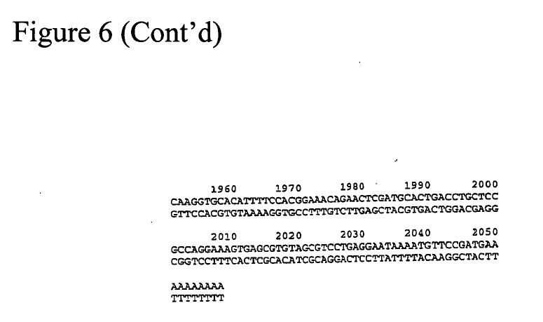

Microinfusion of ZIP into the dorsal hippocampus 3 days after CTA did not impair CTA memory when tested a day after ZIP administration (FIG. 5). If at all, there was a trend toward enhancement of memory. (Repeated-measures ANOVA, (F(1,16)=2.98, p=0.1). Besides demonstrating that PKMζ in the hippocampus is not essential for long-term CTA memory, these data also demonstrate that the effect of ZIP on memory in the insular cortex is region-specific.

There is no evidence that the effect of ZIP on associative taste memory in the IC is reversible through spontaneous recovery or UCS-reinstatement, hence it can be concluded that the PKMζ inhibitor effectively erases the CTA memory in the cortex.

1. A method for erasure or disruption of memory in an animal comprising the administration of a therapeutically effective amount of PKMζ inhibitory peptide.

2. The method of claim 1, wherein the erasure is selective.

3. The method of claim 1, wherein the memory is a long-term associative memory in cortex.

4-5. (canceled)

6. A method of decreasing synaptic transmission in an animal suffering from drug or alcohol addiction, post-traumatic disorder, and phobia, comprising the administration of a therapeutically effective amount of a PKMζ inhibitor.

7. The method of claim 6 wherein said PKMζ inhibitor is chelerythrine.

8. The method of claim 7 wherein said PKMζ inhibitor is myristolated zeta inhibitory pseudosubstrate peptide.

9. The method of claim 1, wherein said animal is a human.

10. The method of claim 2, wherein said animal is a human.

11. The method of claim 3, wherein said animal is a human.

12. The method of claim 6, wherein said animal is a human.

13. The method of claim 7, wherein said animal is a human.

14. The method of claim 8, wherein said animal is a human.
